(19) United States
(12) Patent Application Publication
(10) Pub. No.: US 2026/0207156 A1
(43) Pub. Date: Jul. 23, 2026

(54) X-RAY IMAGE PROCESSING METHOD AND APPARATUS, ELECTRONIC DEVICE AND STORAGE MEDIUM

(71) Applicant: GE Precision Healthcare LLC, Waukesha, WI (US)

(72) Inventors: Han Kang, Beijing (CN); Aizhen Zhou, Beijing (CN); Dejun Wang, Beijing (CN)

(21) Appl. No.: 19/446,586

(22) Filed: Jan. 12, 2026

(30) Foreign Application Priority Data
Jan. 13, 2025 (CN) 202510051871.4

(51) Int. Cl. A61B 6/00; G06T 3/4038; G06T 5/10; G06T 5/50; G06T 5/70

(52) U.S. Cl. CPC A61B 6/5241; A61B 6/5258; G06T 3/4038; G06T 5/10; G06T 5/50; G06T 5/70; G06T 2207/10116; G06T 2207/10144; G06T 2207/20056; G06T 2207/20221

(57) ABSTRACT

The present disclosure relates to the field of image processing, and provides an X-ray image processing method and apparatus, an electronic device, and a storage medium. The method includes: performing a plurality of exposures on at least one body part of a target subject by using an X-ray imaging system to obtain a plurality of first X-ray sub-images; and performing image post-processing on the plurality of first X-ray sub-images to obtain a medical image, where the image post-processing includes separately processing the plurality of first X-ray sub-images by using a virtual grid and performing image stitching, or includes performing image stitching on the plurality of first X-ray sub-images and processing a stitched image by using a virtual grid, and the virtual grid is used to filter out noise caused by scattered radiation in the images.

### CROSS-REFERENCE TO RELATED APPLICATIONS

This application is based on and claims priority to Chinese Patent Application No. 202510051871.4, filed on Jan. 13, 2025, the entire contents of which is herein incorporated by reference.

### TECHNICAL FIELD

The present disclosure relates to the field of image processing, and in particular, to an X-ray image processing method and apparatus, an electronic device, and a storage medium.

### BACKGROUND

X-ray images, also referred to as X-ray films, are technology that uses the penetrating property of X-rays to image an internal structure of a human body or an object. The X-ray images are widely used in the medical field, and doctors can observe structural information, such as pathological conditions in bones, lungs, and abdomen, among other parts, inside a patient's body by using the X-ray images, so as to diagnose diseases such as bone fractures, pneumonia, and tumors. In addition, the X-ray images may also be used to screen for potential health issues such as osteoporosis and tuberculosis.

When the X-rays irradiate the human body, scattering occurs, and scattered radiation is generated. Without processing, noise appears in the obtained X-ray image, resulting in quality degradation of the X-ray image, such as reduced resolution and lowered contrast. In the existing technology, a physical grid is generally disposed on an X-ray imaging device to filter out scattered radiation. However, a scattering degree of the X-ray is related to the thickness of the body part irradiated by the X-ray, and a degree by which scattered radiation is filtered out by the grid is related to parameters of the grid such as the thickness and a gap. Therefore, before exposure starts, the parameters of the grid need to be adjusted based on the thickness of the body part that needs to be examined, and sometimes the grid needs to be disassembled. This makes the steps of obtaining the X-ray image more complex. In an image stitching scenario in which a plurality of X-ray images need to be obtained, it may be necessary to repeatedly adjust the grid or even repeatedly disassemble and assemble the grid. In this case, device costs, labor costs, and time costs in obtaining the X-ray image are all high. After the X-ray image is obtained, the X-ray image needs to be post-processed to obtain a medical image. This increases the device costs, the labor costs, and the time costs in obtaining the medical image in a different form.

### SUMMARY OF THE INVENTION

In view of this, the present disclosure provides an X-ray image processing method and apparatus, an electronic device, and a storage medium. In the method, a virtual grid is used to process an image, achieving a function similar to that of a physical grid, which can reduce device costs brought by the physical grid, thereby reducing device costs, labor costs, and time costs in obtaining a medical image.

According to an aspect of the present disclosure, provided is an X-ray image processing method. The method comprises: performing a plurality of exposures on at least one body part of a target subject by using an X-ray imaging system to obtain a plurality of first X-ray sub-images; and performing image post-processing on the plurality of first X-ray sub-images to obtain a medical image, wherein the image post-processing comprises separately processing the plurality of first X-ray sub-images by using a virtual grid and performing image stitching, or comprises performing image stitching on the plurality of first X-ray sub-images and processing a stitched image by using a virtual grid, and the virtual grid is used to filter out noise caused by scattered radiation in the images.

In a possible implementation, the using a virtual grid comprises processing the first X-ray sub-images or the stitched image based on a scatter image, where the scatter image is obtained by performing Fourier transform filtering on the first X-ray sub-images or the stitched image.

In a possible implementation, the using a virtual grid further comprises adjusting, based on an adjustment parameter, contrast of an image obtained through processing by using the virtual grid.

In a possible implementation, the image post-processing further comprises: separately performing exposure amount adjustment and downsampling on each first X-ray sub-image to obtain a corresponding second X-ray sub-image, and then separately processing the plurality of second X-ray sub-images by using a virtual grid and performing image stitching, or performing image stitching on the plurality of second X-ray sub-images and processing a stitched image by using a virtual grid.

In a possible implementation, the image post-processing further comprises: separately performing image enhancement on the stitched image and each second X-ray sub-image to obtain the medical image.

In a possible implementation, the image enhancement comprises at least one of vertical equalization, multi-resolution enhancement, tissue equalization, inverse gamma distribution transformation, smart windowing, and lookup table mapping.

In a possible implementation, after the performing image stitching on the plurality of first X-ray sub-images and processing a stitched image by using a virtual grid, the image post-processing further comprises performing denoising on the plurality of first X-ray sub-images based on a scatter image, where the scatter image is obtained by performing Fourier transform filtering on the stitched image.

In a possible implementation, the denoising comprises: removing, based on a relationship between any second X-ray sub-image and a first region that is in the stitched image and that corresponds to the second X-ray sub-image, and a second region that is in the scatter image and that corresponds to the second X-ray sub-image, noise caused by scattered radiation in the second X-ray sub-image, wherein the second X-ray sub-image is obtained by performing exposure amount adjustment and downsampling on the first X-ray sub-images.

According to another aspect of the present disclosure, provided is an X-ray image processing apparatus. The apparatus comprises: a first obtaining module, configured to perform a plurality of exposures on at least one body part of a target subject by using an X-ray imaging system to obtain a plurality of first X-ray sub-images; and a second obtaining module, configured to perform image post-processing on the plurality of first X-ray sub-images to obtain a medical image, wherein the image post-processing comprises separately processing the plurality of first X-ray sub-images by using a virtual grid and performing image stitching, or comprises performing image stitching on the plurality of first X-ray sub-images and processing a stitched image by using a virtual grid, and the virtual grid is used to filter out noise caused by scattered radiation in the images.

In a possible implementation, the using a virtual grid comprises processing the first X-ray sub-images or the stitched image based on a scatter image, where the scatter image is obtained by performing Fourier transform filtering on the first X-ray sub-images or the stitched image.

In a possible implementation, the using a virtual grid further comprises adjusting, based on an adjustment parameter, contrast of an image obtained through processing by using the virtual grid.

In a possible implementation, the image post-processing further comprises: separately performing exposure amount adjustment and downsampling on each first X-ray sub-image to obtain a corresponding second X-ray sub-image, and then separately processing the plurality of second X-ray sub-images by using a virtual grid and performing image stitching, or performing image stitching on the plurality of second X-ray sub-images and processing a stitched image by using a virtual grid.

In a possible implementation, the image post-processing further comprises: separately performing image enhancement on the stitched image and each second X-ray sub-image to obtain the medical image.

In a possible implementation, the image enhancement comprises at least one of vertical equalization, multi-resolution enhancement, tissue equalization, inverse gamma distribution transformation, smart windowing, and lookup table mapping.

In a possible implementation, after the performing image stitching on the plurality of first X-ray sub-images and processing a stitched image by using a virtual grid, the image post-processing further comprises performing denoising on the plurality of first X-ray sub-images based on a scatter image, where the scatter image is obtained by performing Fourier transform filtering on the stitched image.

In a possible implementation, the denoising comprises: removing, based on a relationship between any second X-ray sub-image and a first region that is in the stitched image and that corresponds to the second X-ray sub-image, and a second region that is in the scatter image and that corresponds to the second X-ray sub-image, noise caused by scattered radiation in the second X-ray sub-image, wherein the second X-ray sub-image is obtained by performing exposure amount adjustment and downsampling on the first X-ray sub-images.

According to another aspect of the present disclosure, provided is an electronic device. The electronic device comprises: a processor; and a memory, configured to store instructions executable by the processor, wherein the processor is configured to implement the above method when executing the instructions stored in the memory.

According to another aspect of the present disclosure, provided is a non-volatile computer-readable storage medium, having computer program instructions stored thereon, wherein the computer program instructions, when executed by a processor, implement the above method.

According to another aspect of the present disclosure, provided is a computer program product, comprising computer-readable code or a non-volatile computer-readable storage medium bearing computer-readable code, wherein when the computer-readable code is run in a processor of an electronic device, the processor in the electronic device performs the above method.

According to the X-ray image processing method in the embodiments of the present disclosure, by performing a plurality of exposures on at least one body part of a target subject by using an X-ray imaging system, a plurality of first X-ray sub-images may be obtained. Image post-processing is performed on the plurality of first X-ray sub-images to obtain a medical image, where the image post-processing comprises separately processing the plurality of first X-ray sub-images by using a virtual grid and performing image stitching, or comprises performing image stitching on the plurality of first X-ray sub-images and processing a stitched image by using a virtual grid. The virtual grid is used to filter out noise caused by scattered radiation in the images. Therefore, the medical image may be an image from which noise caused by scattered radiation is removed. The virtual grid achieves a function similar to that of a physical grid. In this case, the physical grid does not need to be disposed. This can reduce device costs brought by the physical grid. There is no need to manually adjust the virtual grid, thereby avoiding image quality degradation caused by improper use of the grid. Because the virtual grid is used during image post-processing, the plurality of first X-ray images can be obtained continuously. This greatly improves efficiency of obtaining the plurality of first X-ray images, and reduces labor costs and time costs. In summary, the X-ray image processing method in the embodiments of the present disclosure can reduce device costs brought by the physical grid, thereby reducing device costs, labor costs, and time costs in obtaining the medical image.

Other features and aspects of the present disclosure will become apparent from the following detailed description of exemplary embodiments provided with reference to the accompanying drawings.

### BRIEF DESCRIPTION OF THE DRAWINGS

The accompanying drawings, incorporated in and constituting a part of the specification, illustrate exemplary embodiments, features, and aspects of the present disclosure together with the specification, and serve to explain the principles of the present disclosure.

FIG. 1 shows an exemplary application scenario of an X-ray image processing method according to an embodiment of the present disclosure.

FIG. 2 is a schematic flowchart of an X-ray image processing method according to an embodiment of the present disclosure.

FIG. 3 shows an example of a plurality of first X-ray sub-images according to an embodiment of the present disclosure.

FIG. 4 shows an example of a medical image obtained according to an embodiment of the present disclosure.

FIG. 5 is a schematic diagram of an implementation of denoising according to an embodiment of the present disclosure.

FIG. 6 shows an example of steps included in image post-processing according to an embodiment of the present disclosure.

FIG. 7 shows another example of steps included in image post-processing according to an embodiment of the present disclosure.

FIG. 8 shows an example of a medical image processed in an X-ray image processing method according to an embodiment of the present disclosure.

FIG. 9 is a schematic diagram of a structure of a region of interest according to an embodiment of the present disclosure.

FIG. 10 is a schematic diagram of a structure of an X-ray image processing apparatus according to an embodiment of the present disclosure.

FIG. 11 is a block diagram of an electronic device according to an embodiment of the present disclosure.

### DETAILED DESCRIPTION

Various exemplary embodiments, features, and aspects of the present disclosure will be described in detail below with reference to the accompanying drawings. The same reference numerals in the drawings represent elements having the same or similar functions. While various aspects of the embodiments are illustrated in the accompanying drawing, the accompanying drawings are not necessarily drawn to scale unless specifically stated.

The word “exemplary” dedicated herein means “used as an example or an embodiment, or illustrative”. Any of the embodiments illustrated herein as “exemplary” is not necessarily interpreted as being superior to or better than other embodiments.

In addition, in order to better illustrate the present disclosure, numerous specific details are given in the detailed description below. It should be understood by those skilled in the art that the present disclosure can be implemented without some of the specific details. In some examples, the methods, means, elements, and circuits well known to those skilled in the art are not described in detail in order to highlight the main idea of the present disclosure.

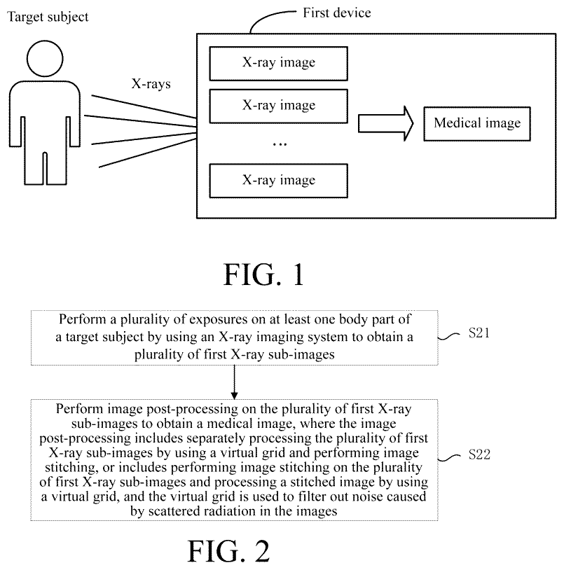

FIG. 1 shows an exemplary application scenario of an X-ray image processing method according to an embodiment of the present disclosure.

As shown in FIG. 1, the X-ray image processing method in the embodiment of the present disclosure may be applied to a first device, and the first device may be an X-ray imaging device, on which an X-ray imaging system (not shown) may be disposed. The first device is not provided with a physical grid.

The first device may perform the X-ray image processing method in the embodiment of the present disclosure, perform a plurality of exposures on at least one body part of a target subject by using the X-ray imaging system to obtain a plurality of X-ray images (for example, first X-ray sub-images described below), and then perform image post-processing on the plurality of X-ray images to obtain a medical image. The first device may present the medical image to a radiologist or output the medical image to a terminal (not shown) used by a radiologist, so that the terminal presents the medical image. The radiologist may analyze a health condition of the target subject based on the medical image.

Those skilled in the art should understand that the first device may alternatively output the obtained plurality of X-ray images to a second device (not shown). The second device may be a terminal device or a server, and the second device performs image post-processing on the plurality of X-ray images to obtain the medical image. The embodiment of the present disclosure imposes no limitation on whether obtaining the X-ray image and performing post-processing on the X-ray image need to be completed by a same device.

Those skilled in the art should understand that performing post-processing on the X-ray image may alternatively be completed by the first device and the second device together. The embodiment of the present disclosure imposes no limitation on steps of image post-processing completed by the first device and the second device respectively.

FIG. 2 is a schematic flowchart of an X-ray image processing method according to an embodiment of the present disclosure.

As shown in FIG. 2, in a possible implementation, the method includes:

Step S21: performing a plurality of exposures on at least one body part of a target subject by using an X-ray imaging system to obtain a plurality of first X-ray sub-images; and

Step S22: performing image post-processing on the plurality of first X-ray sub-images to obtain a medical image, where the image post-processing includes separately processing the plurality of first X-ray sub-images by using a virtual grid and performing image stitching, or includes performing image stitching on the plurality of first X-ray sub-images and processing a stitched image by using a virtual grid, and the virtual grid is used to filter out noise caused by scattered radiation in the images.

For example, body parts of the target subject that need to be exposed may be first determined based on an examination requirement of the target subject. For example, when the target subject wants to examine the right leg, the body part to be exposed may include the right hip, thigh, knee, lower leg, ankle, and foot. For another example, when the target subject wants to examine the right ankle, the body part to be exposed may include the right ankle alone, and other body parts do not need to be exposed.

Step S21 may be performed to perform a plurality of exposures on at least one body part of a target subject by using an X-ray imaging system to obtain a plurality of first X-ray sub-images. When a plurality of body parts of the target subject are exposed, each part may be exposed once or a plurality of times. When one body part of the target subject is exposed, the body part may be exposed a plurality of times. One first X-ray sub-image may be obtained for each exposure, and therefore a plurality of first X-ray sub-images may be obtained after a plurality of exposures.

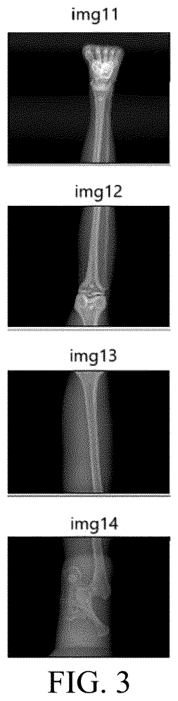

FIG. 3 shows an example of a plurality of first X-ray sub-images according to an embodiment of the present disclosure.

As shown in FIG. 3, a quantity of exposures and a range of each exposure may be preset. For example, the quantity of exposures and the range of each exposure may be determined based on a size of a detector and a body part to be exposed (namely, a exposure region). Specifically, a start position and an end position of the exposure region may be set with the aid of an optical image obtained by a camera, where a preset overlapping portion may be included between ranges of two adjacent exposures

Specifically, for example, the right ankle and foot may be exposed at the first time to obtain an image img11. The right knee and lower leg may be exposed at the second time to obtain an image img12. The right thigh may be exposed at the third time to obtain an image img13. The right hip may be exposed at the fourth time to obtain an image img14.

When some body parts are exposed, a portion (namely, the preset overlapping portion) of adjacent body parts may also appear in the obtained first X-ray sub-images. For example, the image img11 is obtained by exposing the ankle and foot, but also includes a portion of the lower leg.

As can be seen from FIG. 3, the image img11 to the image img14 already include all body parts of the right leg of the target subject, and therefore the exposure may be ended after the image img14 is obtained.

Those skilled in the art should understand that the embodiment of the present disclosure imposes no limitation on a specific quantity of exposures and a specific body part to be exposed in each exposure, provided that the obtained plurality of first X-ray sub-images may include all body parts of the target subject that need to be examined.

Next, step S22 may be performed to perform image post-processing on the plurality of first X-ray sub-images to obtain a medical image. Because no physical grid is used when the first X-ray sub-images are obtained, image post-processing may include processing the images by using the virtual grid, and the virtual grid may simulate a function of the physical grid by using an algorithm to filter out noise caused by scattered radiation in the images. Therefore, the medical image finally obtained after the virtual grid is used during image post-processing has an effect similar to that of a medical image obtained in the existing technology by performing image post-processing by using a physical grid and without using a virtual grid during image exposure.

To improve an image presentation effect, the plurality of first X-ray sub-images may be stitched during image post-processing, so that one stitched image can include all exposed body parts. Image processing may be separately performed using the virtual grid on the plurality of first X-ray sub-images before stitching, or image processing may be performed using the virtual grid on the image obtained by stitching the plurality of first X-ray sub-images. The embodiment of the present disclosure imposes no limitation on a sequence of image stitching and image processing performed by using the virtual grid during image post-processing, provided that the finally obtained medical image includes an image obtained through stitching and processing by using the virtual grid.

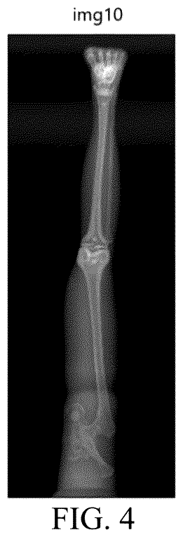

FIG. 4 shows an example of a medical image obtained according to an embodiment of the present disclosure.

As shown in FIG. 4, an image img10 may be a medical image obtained by performing image post-processing on the image img11 to the image img14. It can be seen that the image img10 already includes all body parts of the right leg of the target subject. It can be seen from FIG. 4 that the image img10 does not exhibit smearing, blurring, and the like, and has relatively high contrast, which can meet a requirement for health assessment.

According to the X-ray image processing method in the embodiments of the present disclosure, by performing a plurality of exposures on at least one body part of a target subject by using an X-ray imaging system, a plurality of first X-ray sub-images may be obtained. Image post-processing is performed on the plurality of first X-ray sub-images to obtain a medical image, where the image post-processing includes separately processing the plurality of first X-ray sub-images by using a virtual grid and performing image stitching, or includes performing image stitching on the plurality of first X-ray sub-images and processing a stitched image by using a virtual grid. The virtual grid is used to filter out noise caused by scattered radiation in the images. Therefore, the medical image may be an image from which noise caused by scattered radiation is removed. The virtual grid achieves a function similar to that of a physical grid. In this case, the physical grid does not need to be disposed. This can reduce device costs brought by the physical grid. There is no need to manually adjust the virtual grid, thereby avoiding image quality degradation caused by improper use of the grid. Because the virtual grid is used during image post-processing, the plurality of first X-ray images can be obtained continuously. This greatly improves efficiency of obtaining the plurality of first X-ray images, and reduces labor costs and time costs. In summary, the X-ray image processing method in the embodiments of the present disclosure can reduce device costs brought by the physical grid, thereby reducing device costs, labor costs, and time costs in obtaining the medical image.

The medical image obtained by using the virtual grid in oblique exposure and defocused exposure scenarios has better quality than that obtained by using a physical grid, and potential residual artifacts and ringing artifacts are avoided, which can further improve image quality.

An example of steps included in image processing performed by using the virtual grid is described below.

In a possible implementation, the using a virtual grid includes processing the first X-ray sub-images or the stitched image based on a scatter image, where the scatter image is obtained by performing Fourier transform filtering on the first X-ray sub-images or the stitched image.

For example, processing the first X-ray sub-images by using the virtual grid may be a process of performing fast Fourier transform (FFT) filtering on the first X-ray sub-images to obtain a scatter image, and then subtracting the scatter image from the first X-ray sub-images, thereby achieving noise removal for the first X-ray sub-images. In this case, the scatter image may include noise caused by scattered radiation in the first X-ray sub-images.

Similarly, processing the stitched image by using the virtual grid may be a process of performing fast Fourier transform (Fast Fourier Transform, FFT) filtering on the stitched image to obtain a scatter image, and then subtracting the scatter image from the stitched image, thereby achieving noise removal for the stitched image. In this case, the scatter image may include noise caused by scattered radiation in the stitched image.

In this way, noise caused by scattered radiation can be filtered out.

In a possible implementation, the using a virtual grid further includes adjusting, based on an adjustment parameter, contrast of an image obtained through processing by using the virtual grid.

For example, the adjustment parameter may be used to adjust contrast of the image, and the adjustment parameter is related to a grid ratio and a grid density of the physical grid when the physical grid is used to obtain images including a same body part. A plurality of levels may be set for the adjustment parameter, and a higher level indicates a greater contrast change caused by the adjustment parameter. For example, a selection or adjustment button for the adjustment parameter may be provided in a graphical user interface, and a user may perform selection or adjustment based on a requirement. In one example, three levels may be preset, which are respectively a high level/a middle level/a low level. The embodiment of the present disclosure imposes no limitation on a quantity of levels of the adjustment parameter and a contrast change degree corresponding to each level.

In a scenario in which the first X-ray sub-images are processed by using the virtual grid, an image obtained after processing by using the virtual grid may be an image after the scatter image is subtracted from the first X-ray sub-images, and the adjustment parameter may be used to adjust contrast of the image. Because body parts included in the image is the same as body parts included in the first X-ray sub-images, a level corresponding to each first X-ray sub-image may be determined based on the body part included in the first X-ray sub-image, and then an adjustment parameter used by the virtual grid is determined based on the levels corresponding to the plurality of first X-ray sub-images. When each first X-ray sub-image is separately processed by using the virtual grid, the virtual grid may use the same adjustment parameter.

For example, the level corresponding to each first X-ray sub-image may be determined first, and if a quantity of first X-ray sub-images corresponding to the high level is the largest, it may be determined that the adjustment parameter used by the virtual grid is the high level. The embodiment of the present disclosure imposes no limitation on a specific implementation of determining, based on the plurality of first X-ray sub-images, the adjustment parameter used by the virtual grid, provided that the determined adjustment parameter can improve quality of some of the first X-ray sub-images. After the scatter image is subtracted from the first X-ray sub-images, the contrast of the image is adjusted by using the adjustment parameter.

Similarly, in a scenario in which the stitched image is processed by using the virtual grid, an image obtained after processing by using the virtual grid may be an image after the scatter image is subtracted from the stitched image, and the adjustment parameter may be used to adjust contrast of the image. Because a body part included in the image is the same as a body part included in the stitched image, an adjustment parameter used by the virtual grid may be determined based on the body part included in the stitched image. Exemplary manners of determining the adjustment parameter have been described above, and details are not described herein again.

In this way, the contrast of the image after processing by using the virtual grid can be improved.

Those skilled in the art should understand that image processing performed by using the virtual grid may alternatively include more or fewer steps, provided that noise caused by scattered radiation can be removed. The embodiment of the present disclosure imposes no limitation on specific steps included in image processing performed by using the virtual grid.

It can be understood that, in addition to image stitching and image processing implemented by using the virtual grid, image post-processing may further include more steps.

In a possible implementation, the image post-processing further includes:

separately performing exposure amount adjustment and downsampling on each first X-ray sub-image to obtain a corresponding second X-ray sub-image, and then separately processing the plurality of second X-ray sub-images by using a virtual grid and performing image stitching, or performing image stitching on the plurality of second X-ray sub-images and processing a stitched image by using a virtual grid.

For example, before the first X-ray sub-images are separately processed by using the virtual grid, exposure amount adjustment (Milliampere Seconds Scaling) and downsampling (Down Sampling) may be performed on the first X-ray sub-images to obtain the corresponding second X-ray sub-images.

Exposure amount adjustment is mainly implemented by adjusting a value of milliampere seconds (mAs) of the image. mAs represents the product of current in an X-ray tube and time, and is an important parameter of an X-ray exposure amount. mAs determines a quantity of X-rays, namely, a total quantity of X-ray photons penetrating an object being imaged. A higher value of mAs produces more X-ray photons, thereby increasing image brightness. Conversely, a lower value of mAs reduces image brightness. In image post-processing, image brightness may be changed by adjusting the value of mAs (namely, performing mAs scaling), thereby improving image quality, and balancing contrast and brightness of the image to optimize an assessment effect. Downsampling may reduce a data amount of the image and reduce image processing costs of subsequent steps.

An manner of exposure amount adjustment and a manner of downsampling may be determined based on a requirement of an application scenario. Both exposure amount adjustment and downsampling may be implemented based on the existing technology, and details of exposure amount adjustment and downsampling are not described herein again.

In this way, an noise removal effect of the virtual grid can be improved, and data processing costs of image processing performed by using the virtual grid can be reduced.

In this case, the plurality of second X-ray sub-images may be separately processed by using the virtual grid and image stitching may be performed, and a specific implementation may be the same as that of separately processing the plurality of first X-ray sub-images by using the virtual grid and performing image stitching. Details are not described herein again. Alternatively, image stitching may be performed on the plurality of second X-ray sub-images, and the stitched image may be processed by using the virtual grid. A specific implementation is the same as that of performing image stitching on the plurality of first X-ray sub-images and processing the stitched image by using the virtual grid. Details are not described herein again.

It can be understood that the first X-ray sub-images may alternatively be processed directly by using the virtual grid, or exposure amount adjustment may be performed on the first X-ray sub-images before being processed by using the virtual grid, and downsampling may be performed on the first X-ray sub-images after being processed by using the virtual grid. The embodiment of the present disclosure imposes no limitation on whether exposure amount adjustment and downsampling are necessary before the first X-ray sub-images are processed by using the virtual grid.

In a possible implementation, after the performing image stitching on the plurality of first X-ray sub-images and processing a stitched image by using a virtual grid, the image post-processing further includes performing denoising on the plurality of first X-ray sub-images based on a scatter image, where the scatter image is obtained by performing Fourier transform filtering on the stitched image.

For example, as described above, when the stitched image is processed by using the virtual grid, a scatter image may be obtained, and the scatter image includes noise caused by scattered radiation in the stitched image. It can be understood that the X-ray sub-images (including the first X-ray sub-images and the second X-ray sub-images obtained through exposure amount adjustment and/or downsampling) also have noise caused by scattered radiation. To improve accuracy of health assessment based on the medical image, after image stitching is performed on the first X-ray sub-images and the stitched image is processed by using the virtual grid, denoising may further be separately performed on the plurality of X-ray sub-images based on the scatter image, and the purpose of denoising is to remove noise caused by scattered radiation. The denoised X-ray sub-images and the stitched image after image enhancement are stored together in a memory. The memory may be a memory in an imaging system, or may be a cloud memory or a medical Picture Archiving and Communication System (PACS). If some body parts still remain unclear in the stitched image after image enhancement, the denoised X-ray sub-images can be checked to improve accuracy of health condition analysis. An exemplary implementation of denoising is described below.

In a possible implementation, the denoising includes:

removing, based on a relationship between any second X-ray sub-image and a first region that is in the stitched image and that corresponds to the second X-ray sub-image, and a second region that is in the scatter image and that corresponds to the second X-ray sub-image, noise caused by scattered radiation in the second X-ray sub-image, where the second X-ray sub-image is obtained by performing exposure amount adjustment and downsampling on the first X-ray sub-images.

Specifically, similar to removing noise in the stitched image, to remove noise in the X-ray sub-images, by subtracting a corresponding scatter sub-image from the X-ray sub-image, a corresponding X-ray sub-image with high image quality can be obtained. Because the scatter image is obtained based on the stitched image, to obtain a corresponding scatter sub-image of each region, it is necessary to obtain a proportional relationship (or a ratio) between the scatter sub-image of each region and the scatter image. Therefore, a relationship between the scatter sub-image of each region and the scatter image can be obtained by using the proportional relationship between each X-ray sub-image and the stitched image, to thereby obtain the scatter sub-image of each region. In some embodiments, the proportional relationship between each X-ray sub-image and the stitched image may be obtained by means of a binary image corresponding to the stitched image.

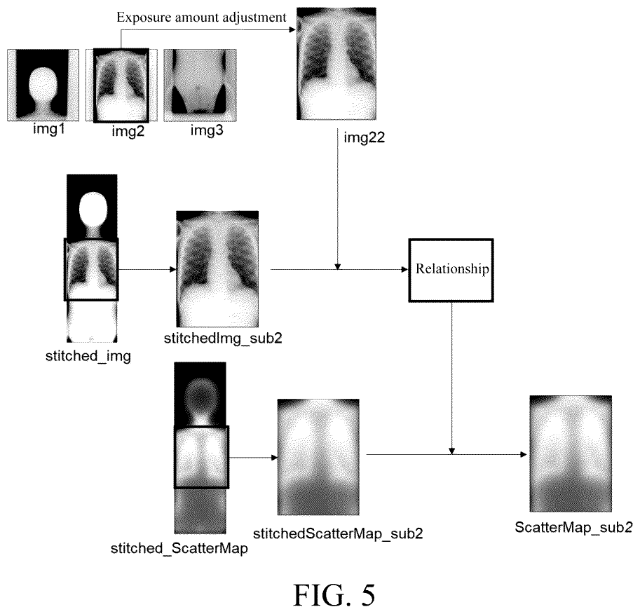

FIG. 5 is a schematic diagram of an implementation of denoising according to an embodiment of the present disclosure.

As shown in FIG. 5, it is assumed that a plurality of first X-ray sub-images are img1 to img3 respectively. An image obtained by stitching the plurality of first X-ray sub-images may be stitched_img. A scatter image may be stitched_ScatterMap.

A second X-ray sub-image may be obtained by performing exposure amount adjustment and downsampling on each first X-ray sub-image. For each second X-ray sub-image, a first region that is in the stitched image and that corresponds to the second X-ray sub-image and a second region that is in the scatter image and that corresponds to the second X-ray sub-image may be extracted. Since the second X-ray sub-image may have the same size as the corresponding first region and second region, an included body part may be the same.

In FIG. 5, the first X-ray sub-image img2 is used as an example. A second X-ray sub-image img22 may be obtained after exposure amount adjustment and downsampling are performed on the image img2. Further, when a plurality of second X-ray sub-images are stitched, only a portion of the second X-ray sub-image img22 may remain in the stitched image. In this case, a size of the image img22 may also be adjusted to be consistent with a size of a portion that is in the stitched image and that belongs to the second X-ray sub-image img22.

The stitched image has a first region stitchedImg_sub2 corresponding to the image img22. Association relationships between pixel values of the image img22 and pixel values of the first region stitchedImg_sub2 may be determined based on the image img22 and the first region stitchedImg_sub2. The scatter image also has a second region stitchedScatterMap_sub2 corresponding to the image img22. When the association relationships between the pixel values of the image img22 and the pixel values of the first region stitchedImg_sub2 are known, an image ScatterMap_sub2 may be obtained, so that same association relationships exist between the image ScatterMap_sub2 and the second region stitchedScatterMap_sub2.

In this case, noise caused by scattered radiation in the image img22 may be removed based on the image ScatterMap_sub2. For example, a pixel value of a corresponding pixel in the image ScatterMap_sub2 is subtracted from a pixel value of each pixel in the image img22.

In this way, when the virtual grid is applied to the stitched image, noise caused by scattered radiation in each second X-ray sub-image may also be removed, and consistency between a noise removal manner of the plurality of second X-ray sub-images and a noise removal manner of the stitched image is maintained.

In a possible implementation, the image post-processing further includes:

separately performing image enhancement on the stitched image and each second X-ray sub-image to obtain the medical image.

For example, after exposure amount adjustment and downsampling are completed, each second X-ray sub-image may be separately processed by using the virtual grid, and the plurality of processed second X-ray sub-images may be stitched. The stitched image may be directly used as the medical image, or image enhancement may be continued to obtain a corresponding medical image. Image enhancement may also be performed on each processed second X-ray sub-image to obtain a corresponding medical image. If some body parts still remain unclear in the medical image obtained after image enhancement on the stitched image, the medical image obtained after image enhancement on the second X-ray sub-images including corresponding body parts may be checked. In this way, health assessment is more accurate.

Those skilled in the art should understand that, if the first X-ray sub-images before exposure amount adjustment and downsampling are processed using the virtual grid during image post-processing, image enhancement may also be separately performed on the processed first X-ray sub-images to obtain the medical image.

In a possible implementation, the image enhancement includes at least one of vertical equalization, multi-resolution enhancement, tissue equalization, inverse gamma distribution transformation, smart windowing, and lookup table mapping.

Vertical equalization (VE) refers to applying an equalization technology in a vertical direction of an image, to enhance contrast of the image.

Vertical equalization may be implemented based on the existing technology. For example, histogram equalization or the like in the existing technology may be used to segment grayscales of the image in the vertical direction, and a grayscale of each segment may be adjusted through equalization. The embodiment of the present disclosure imposes no limitation on an equalization technique specifically used in the vertical equalization step, provided that image quality can be enhanced.

Multi-resolution (MR) enhancement is a method of enhancing an image by using a multi-resolution analysis technology. Multi-resolution analysis is technology for decomposing an image into different scales and frequency components, and useful information in the image can be effectively extracted and enhanced by processing the image at different resolution levels. Commonly used multi-resolution enhancement technology include wavelets transform, pyramid decomposition, and the like. Multi-resolution enhancement may be implemented based on the existing technology. Details of a specific implementation of multi-resolution enhancement are not described herein again.

Tissue equalization (Tissue Equalization, TE) is image processing technology, which decomposes an image into regions with different densities, separately processes the regions, and then performs weighted integration on these images to obtain a new image. This processing technology enables tissues with different densities to be well displayed in the final image, thereby improving contrast and clarity of the image, and helping doctors to perform more accurate diagnosis and treatment. Tissue equalization may be implemented based on the existing technology. Details of a specific implementation of tissue equalization are not described herein again.

Inverse gamma (Inverse Gamma) distribution transformation may be used for image enhancement and restoration. Inverse gamma distribution transformation may use its characteristics of probability density function to perform non-linear transformation on the image, thereby improving contrast and clarity of the image. Specifically, inverse gamma distribution transformation may control a grayscale distribution of the image by adjusting its shape parameter and scale parameter. When the overall image is dark or bright, the grayscale distribution of the image may be made more uniform by selecting an appropriate parameter value, thereby improving a visual effect of the image. In addition, inverse gamma distribution transformation may also be used for denoising the image, to further improve image quality by reducing an impact of noise on the image. Inverse gamma distribution transformation may be implemented based on the existing technology. Details of a specific implementation of inverse gamma distribution transformation are not described herein again.

Smart windowing, also referred to as window width and window level adjustment, is technology for enhancing contrast and brightness of an image by adjusting a grayscale range of the image. In medical image processing, because a grayscale range of a medical image is generally very wide (possibly up to thousands of grayscales), while a grayscale range of a display is limited (generally 0 to 255), it is necessary to selectively display a grayscale range of interest through smart windowing. A window width and a window level (also referred to as a window center) are two key parameters in smart windowing. The window width defines a range of grayscales displayed in the image, and the window level determines a center value of the range. These two parameters are adjusted, so that contrast and brightness of the image can be changed, thereby highlighting specific tissue structures or lesions. Smart windowing may be implemented based on the existing technology. Details of a specific implementation of smart windowing are not described herein again.

Lookup table (LUT) mapping is technology for quickly converting an input value into an output value by using a predefined mapping relationship. In image processing, a LUT is generally used to map a pixel value (for example, an RGB value) of an image into a new pixel value to achieve a particular visual effect.

A working principle of the LUT is based on a predefined array or table, and the predefined array or table stores a mapping relationship between an input value and an output value. When the image needs to be transformed, an output value can be quickly obtained by simply looking up the LUT for a corresponding input value. In image processing, the LUT is generally a three-dimensional array that is used to store all possible input color values in an RGB color space and the corresponding output color values. In this way, for each pixel in the image, a new color value of the pixel can be obtained by looking up the LUT.

Lookup table mapping may be implemented based on the existing technology. Details of a specific implementation of lookup table mapping are not described herein again.

Those skilled in the art should understand that image enhancement may alternatively include more or fewer steps, provided that quality of the processed image can be improved. The embodiment of the present disclosure imposes no limitation on steps specifically included in image enhancement.

In this way, quality of the medical image may be further improved, and the manner of improving quality of the medical image is more flexible.

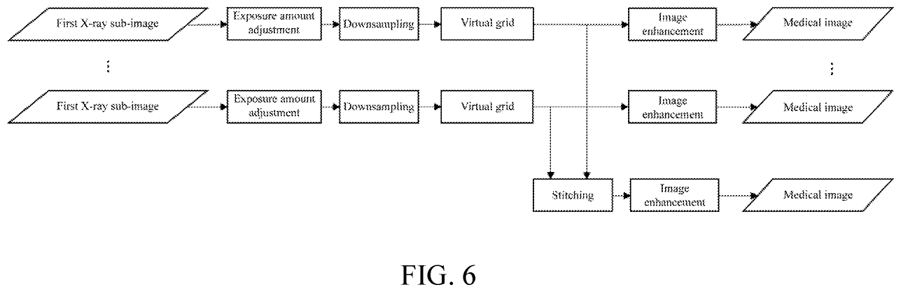

FIG. 6 shows an example of steps included in image post-processing according to an embodiment of the present disclosure.

As shown in FIG. 6, performing image post-processing on the first X-ray sub-images may include the following steps: First, exposure amount adjustment and downsampling are performed on each first X-ray sub-image, and then the downsampled image is processed by using the virtual grid. The images processed by using the virtual grid are stitched, and image enhancement is performed on the stitched image to obtain the medical image. Image enhancement is separately performed on the images processed by using the virtual grid to obtain the medical image. A specific implementation of each step has been described above, and details are not described herein again.

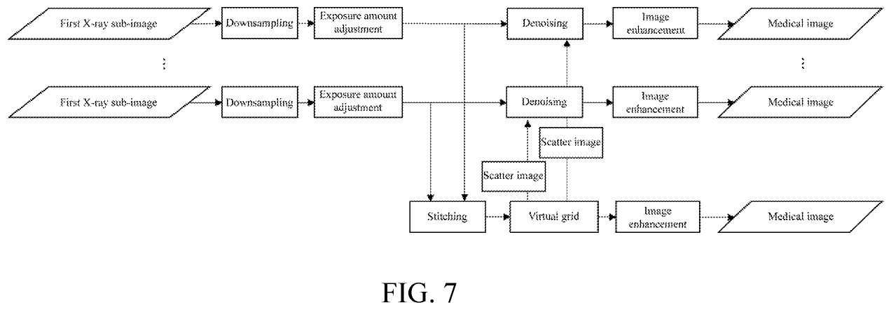

FIG. 7 shows another example of steps included in image post-processing according to an embodiment of the present disclosure.

As shown in FIG. 7, performing image post-processing on the first X-ray sub-images may include the following steps: First, downsampling and exposure amount adjustment are performed on each first X-ray sub-image, and then the images after exposure amount adjustment are stitched. The stitched image is processed by using the virtual grid. Image enhancement is performed on the image processed by using the virtual grid to obtain the medical image. When the stitched image is processed by using the virtual grid, a scatter image corresponding to the stitched image is also generated, and based on the scatter image and the stitched image, denoising may be performed on the images after exposure amount adjustment. Image enhancement is separately performed on the denoised images to obtain the medical image. A specific implementation of each step has been described above, and details are not described herein again.

Those skilled in the art should understand that image post-processing may alternatively include more or fewer steps, provided that image post-processing includes separately processing the plurality of first X-ray sub-images by using the virtual grid and performing image stitching, or includes performing image stitching on the plurality of first X-ray sub-images and processing the stitched image by using the virtual grid. The embodiment of the present disclosure imposes no limitation on specific steps included in image post-processing.

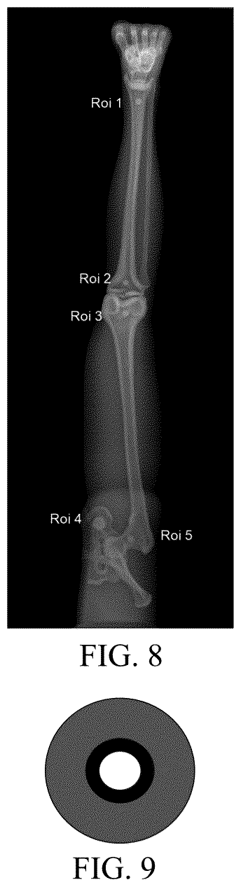

FIG. 8 shows an example of a medical image processed in an X-ray image processing method according to an embodiment of the present disclosure. A quality evaluation method and a quality evaluation result of the medical image shown in FIG. 8 are given below.

As shown in FIG. 8, the medical image obtained by using the X-ray image processing method according to the embodiment of the present disclosure is an X-ray image of a right leg, and five regions of interest (Roi 1 to Roi 5) may be set on the medical image. FIG. 9 is a schematic diagram of a structure of a region of interest according to an embodiment of the present disclosure. The region of interest is in an annular shape, namely, a black portion in FIG. 9. An average pixel value of a circular region inside the region of interest is used as a signal value of the region of interest, and an average pixel value of a circular region outside the region of interest is used as a background value of the region of interest.

The right leg of the target subject may be exposed by using an X-ray imaging device provided with a physical grid in the existing technology, to obtain a plurality of sample X-ray sub-images and stitch same to obtain a sample medical image. Five regions of interest are also set at the same positions on the sample medical image.

Quality of the medical image may be evaluated by using two indicators, a relative contrast improvement factor Rel.CIF and a relative contrast-to-noise ratio Rel.CNR.

An example of a manner of calculating the relative contrast improvement factor is given below.

For each of the sample medical image and the medical image in the embodiment of the present disclosure, a difference between the background value and the signal value of each region of interest is calculated, and then a ratio of the difference to the background value is calculated to obtain contrast of the region of interest. The contrast of the region of interest in the sample medical image may be CR_Grid, and the contrast of the region of interest in the medical image in the embodiment of the present disclosure may be CR_AG.

A difference CR AG-CR Grid between the contrast of the region of interest in the medical image in the embodiment of the present disclosure and the contrast of the region of interest in the sample medical image may be calculated, and then a ratio (CR AG-CR_Grid)/CR_Grid of the difference to the contrast of the region of interest in the sample medical image may be calculated to obtain the relative contrast improvement factor Rel.CIF of the region of interest in the medical image in the embodiment of the present disclosure.

An example of a manner in which the relative contrast-to-noise ratio is calculated is given below.

A standard deviation Sigma of pixel values of a region of interest at a same position of the medical image in the embodiment of the present disclosure and the sample medical image may be calculated. A difference between the background value and the signal value of the region of interest in the medical image in the embodiment of the present disclosure may be calculated, and a ratio of the difference to the standard deviation is used as the contrast-to-noise ratio CNR_AG of the region of interest in the medical image in the embodiment of the present disclosure. A difference between the background value and the signal value of the region of interest in the sample medical image may be calculated, and a ratio of the difference to the standard deviation is used as the contrast-to-noise ratio CNR_Grid of the region of interest in the sample medical image. A difference between CNR AG and CNR Grid is calculated, and then a ratio of the difference to CNR_Grid, namely, (CNR_AG-CNR_Grid)/CNR_Grid, is calculated as the relative contrast-to-noise ratio Rel.CNR of the region of interest in the medical image in the embodiment of the present disclosure.

Table 1 shows the relative contrast improvement factor Rel.CIF of each region of interest in the medical image obtained when the first X-ray sub-image is used as an object used by the virtual grid in the image post-processing process in the X-ray image processing method in the embodiment of the present disclosure.

TABLE 1

| | Relative contrast |
|---|---|
| Region | improvement |
| of interest | factor, Rel. CIF |
| Roi 1 | 0.05372 |
| Roi 2 | 0.198624 |
| Roi 3 | 0.384295 |
| Roi 4 | −0.03399 |
| Roi 5 | −0.12747 |

It can be seen from Table 1 that the relative contrast improvement factors Rel.CIF of 60% of the regions of interest in the medical image obtained in the embodiment of the present disclosure all have positive values. Therefore, quality of the medical image obtained in the embodiment of the present disclosure is better than that of the sample medical image in the existing technology.

It can be learned from FIG. 3 and FIG. 8, the region of interest Roi 1 belongs to the first X-ray sub-image img11, the regions of interest Roi 2 and Roi 3 belong to the first X-ray sub-image img12, and the regions of interest Roi 4 and Roi 5 belong to the first X-ray sub-image img14. Table 2 to Table 4 also show the relative contrast improvement factor Rel. CIF of the region of interest in each first X-ray sub-image after undergoing image enhancement. In this case, the first X-ray sub-images obtained in the existing technology may be subjected to image enhancement and then used as the sample medical image. A calculation manner of the relative contrast improvement factor Rel.CIF has been described above, and details are not described herein again.

TABLE 2

| | Relative contrast |
|---|---|
| Region | improvement |
| of interest | factor, Rel. CIF |
| Roi 1 | 0.049357 |

TABLE 3

| | Relative contrast |
|---|---|
| Region | improvement |
| of interest | factor, Rel. CIF |
| Roi 2 | 0.197488 |
| Roi 3 | 0.358113 |

TABLE 4

| | Relative contrast |
|---|---|
| Region | improvement |
| of interest | factor, Rel. CIF |
| Roi 4 | −0.04265 |
| Roi 5 | −0.12217 |

It can be seen from Table 2 to Table 4 that the relative contrast improvement factors Rel.CIF of the regions of interest in most of the first X-ray sub-images after undergoing image enhancement all have positive values. Therefore, quality of the first X-ray sub-images, after undergoing image enhancement, that are obtained in the embodiment of the present disclosure is highly probable to be better than that of the sample medical image in the existing technology.

Table 5 shows the relative contrast improvement factor Rel.CIF of each region of interest in the medical image obtained when the stitched image is used as an object used by the virtual grid in the image post-processing process in the X-ray image processing method in the embodiment of the present disclosure.

TABLE 5

| | Relative contrast |
|---|---|
| Region | improvement |
| of interest | factor, Rel. CIF |
| Roi 1 | 0.023902 |
| Roi 2 | 0.144857 |
| Roi 3 | 0.403847 |
| Roi 4 | 0.199675 |
| Roi 5 | −0.12444 |

It can be seen from Table 5 that the relative contrast improvement factors Rel.CIF of 80% of the regions of interest in the medical image obtained in the embodiment of the present disclosure all have positive values. Therefore, quality of the medical image obtained in the embodiment of the present disclosure is better than that of the sample medical image in the existing technology.

The region of interest Roi 1 belongs to the first X-ray sub-image img1, the regions of interest Roi 2 and Roi 3 belong to the first X-ray sub-image img2, and the regions of interest Roi 4 and Roi 5 belong to the first X-ray sub-image img4. Table 6 to Table 8 also show the relative contrast improvement factor Rel. CIF of the region of interest in each first X-ray sub-image after undergoing image enhancement. In this case, the first X-ray sub-images obtained in the existing technology may be subjected to image enhancement and then used as the sample medical image. A calculation manner of the relative contrast improvement factor Rel.CIF has been described above, and details are not described herein again.

TABLE 6

| | Relative contrast |
|---|---|
| Region | improvement |
| of interest | factor, Rel. CIF |
| Roi 1 | 0.024129 |

TABLE 7

| | Relative contrast |
|---|---|
| Region | improvement |
| of interest | factor, Rel. CIF |
| Roi 2 | 0.14922 |
| Roi 3 | 0.397485 |

TABLE 8

| | Relative contrast |
|---|---|
| Region | improvement |
| of interest | factor, Rel. CIF |
| Roi 4 | 0.184639 |
| Roi 5 | −0.12113 |

It can be seen from Table 6 to Table 8 that the relative contrast improvement factors Rel.CIF of the regions of interest in most of the first X-ray sub-images after undergoing image enhancement all have positive values. Therefore, quality of the first X-ray sub-images, after undergoing image enhancement, that are obtained in the embodiment of the present disclosure is highly probable to be better than that of the sample medical image in the existing technology.

An embodiment of the present disclosure further provides an X-ray image processing apparatus. FIG. 10 is a schematic diagram of a structure of an X-ray image processing apparatus according to an embodiment of the present disclosure.

As shown in FIG. 10, in a possible implementation, the apparatus includes:
a first obtaining module 10, configured to perform a plurality of exposures on at least one body part of a target subject by using an X-ray imaging system to obtain a plurality of first X-ray sub-images; and
a second obtaining module 11, configured to perform image post-processing on the plurality of first X-ray sub-images to obtain a medical image, where the image post-processing includes separately processing the plurality of first X-ray sub-images by using a virtual grid and performing image stitching, or includes performing image stitching on the plurality of first X-ray sub-images and processing a stitched image by using a virtual grid, and the virtual grid is used to filter out noise caused by scattered radiation in the images.

In a possible implementation, the using a virtual grid includes processing the first X-ray sub-images or the stitched image based on a scatter image, where the scatter image is obtained by performing Fourier transform filtering on the first X-ray sub-images or the stitched image.

In a possible implementation, the using a virtual grid further includes adjusting, based on an adjustment parameter, contrast of an image obtained through processing by using the virtual grid.

In a possible implementation, the image post-processing further includes: separately performing exposure amount adjustment and downsampling on each first X-ray sub-image to obtain a corresponding second X-ray sub-image, and then separately processing the plurality of second X-ray sub-images by using a virtual grid and performing image stitching, or performing image stitching on the plurality of second X-ray sub-images and processing a stitched image by using a virtual grid.

In a possible implementation, the image post-processing further includes: separately performing image enhancement on the stitched image and each second X-ray sub-image to obtain the medical image.

In a possible implementation, the image enhancement includes at least one of vertical equalization, multi-resolution enhancement, tissue equalization, inverse gamma distribution transformation, smart windowing, and lookup table mapping.

In a possible implementation, after the performing image stitching on the plurality of first X-ray sub-images and processing a stitched image by using a virtual grid, the image post-processing further includes performing denoising on the plurality of first X-ray sub-images based on a scatter image, where the scatter image is obtained by performing Fourier transform filtering on the stitched image.

In a possible implementation, the denoising includes: removing, based on a relationship between any second X-ray sub-image and a first region that is in the stitched image and that corresponds to the second X-ray sub-image, and a second region that is in the scatter image and that corresponds to the second X-ray sub-image, noise caused by scattered radiation in the second X-ray sub-image, wherein the second X-ray sub-image is obtained by performing exposure amount adjustment and downsampling on the first X-ray sub-images.

In some embodiments, a function of the apparatus provided in the embodiment of the present disclosure or the modules included in the apparatus may be configured to perform the method described in the foregoing method embodiments. For a specific implementation thereof, reference may be made to descriptions of the foregoing method embodiments. For brevity, details are not described herein again.

An embodiment of the present disclosure further provides a computer-readable storage medium, having computer program instructions stored thereon, wherein the computer program instructions, when executed by a processor, implement the above method. The computer-readable storage medium may be a volatile or non-volatile computer-readable storage medium.

An embodiment of the present disclosure further provides an electronic device, including: a processor; a memory, configured to store instructions executable by the processor, wherein the processor is configured to implement the above method when executing the instructions stored in the memory.

An embodiment of the present disclosure further provides a computer program product, including computer-readable code or a non-volatile computer-readable storage medium bearing computer-readable code, wherein when the computer-readable code is run in a processor in an electronic device, the processor in the electronic device performs the above method.

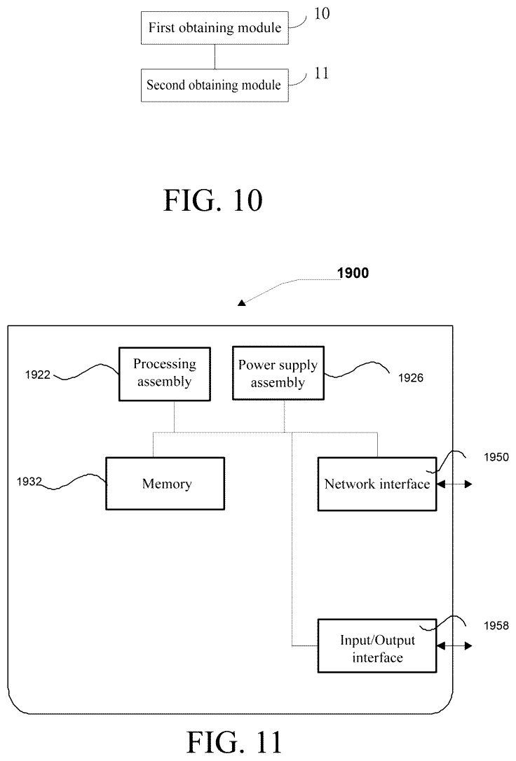

FIG. 11 is a block diagram of an electronic device according to an embodiment of the present disclosure. For example, the electronic device 1900 may be provided as a server or a terminal device. Referring to FIG. 11, the electronic device 1900 includes a processing assembly 1922 that further includes one or more processors, and a memory resource represented by a memory 1932 for storing instructions that can be executed by the processing assembly 1922, such as an application. The application stored in the memory 1932 can include one or more modules each corresponding to a set of instructions. In addition, the processing assembly 1922 is configured to execute instructions to execute the above method.

The electronic device 1900 can further include a power supply assembly 1926 configured to perform power management of the electronic device 1900, a wired or wireless network interface 1950 configured to connect the electronic device 1900 to a network, and an input/output interface (I/O interface) 1958. The electronic device 1900 may operate based on an operating system stored in the memory 1932, for example, Windows Server™, Mac OS X™, Unix™, Linux™, or FreeBSD™.

In an exemplary embodiment, a non-volatile computer-readable storage medium is also provided, such as the memory 1932 including computer program instructions that are executable by the processing assembly 1922 of the electronic device 1900 to perform the above method.

The present disclosure may be a system, a method and/or a computer program product. The computer program product may include a computer-readable storage medium, having computer-readable program instructions thereon for causing a processor to implement various aspects of the present disclosure.

The computer-readable storage medium can be a tangible device that can hold and store instructions used by an instruction execution device. The computer-readable storage medium may be, for example, but is not limited to an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read only memory (ROM), a erasable programmable read only memory (EPROM or flash memory), a static random access memory (SRAM), a portable compact disc read-only memory (CD-ROM), a digital versatile disk (DVD), a memory stick, a floppy disk, mechanical coding equipment, such as a punch card having instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers, and/or edge servers. A network adapter card or a network interface in each computing/processing device receives computer-readable program instructions from the network and forwards the computer-readable program instructions, for storing them in a computer-readable storage medium in each computing/processing device.

Computer program instructions for executing the operations of the present disclosure can be assembly instructions, instruction set architecture (ISA) instructions, machine instructions, machine related instructions, microcode, firmware instructions, state setting data, or source code or object code written in any combination of one or more programming languages, the programming language including object oriented programming languages such as Smalltalk, C++ and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to the user's computer through any kind of network, including a local area network (LAN) or a wide area network (WAN). Alternatively, it can be connected to an external computer (for example, using an Internet service provider to connect via the Internet). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure.

The aspects of the present disclosure are described herein with reference to the flowcharts and/or block diagrams of the methods, apparatuses (systems), and computer program products according to the embodiments of the present disclosure. It should be understood that each block of the flowcharts and/or block diagrams and combinations of various blocks in the flowcharts and/or block diagrams can be implemented by computer-readable program instructions.

These computer-readable program instructions may be provided to a processor of a general-purpose computer, a special-purpose computer, or other programmable data processing apparatuses, to produce a machine, so that these instructions, when executed by the processor of the computer or other programmable data processing apparatuses, produce an apparatus for implementing the functions/actions specified in one or more blocks of the flowcharts and/or block diagrams. Also, these computer-readable program instructions may be stored in a computer-readable storage medium. These instructions cause a computer, a programmable data processing apparatus, and/or other devices to work in a specific manner. Thus, the computer-readable medium storing the instructions includes an artifact, including instructions that implement various aspects of the functions/actions specified in one or more the flowcharts and/or block diagrams.

The computer-readable program instructions may also be loaded onto a computer, other programmable data processing apparatuses, or other devices, such that the computer, other programmable data processing apparatuses or other devices execute a series of operational steps, to generate a computer-implemented process, such that the functions/actions specified in one or more of the flowcharts and/or block diagrams are implemented by the instructions executed on the computer, other programmable data processing apparatuses, or other devices.

The flowcharts and block diagrams in the accompanying drawings illustrate system architectures, functions, and operations of possible implementations of the system, method, and computer program product according to a plurality of embodiments of the present disclosure. In this regard, each block in the flowcharts or block diagrams may represent a portion of a module, program segment, or instruction that contains one or more executable instructions for implementing the specified logical functions. In some alternative implementations, the functions denoted in the blocks can also occur in a different order than that illustrated in the drawings. For example, two consecutive blocks can actually be executed substantially in parallel, and sometimes can also be executed in a reverse order, depending upon the functions involved. It should also be noted that each block of the block diagrams and/or flowcharts, and combinations of blocks in the block diagrams and/or flowcharts can be implemented in a dedicated hardware-based system that executes the specified function or action, or can be implemented by a combination of dedicated hardware and computer instructions.

The embodiments of the present disclosure have been described above. The foregoing description is illustrative rather than limiting, and is not limited to the disclosed embodiments. Many modifications and variations are apparent to those of ordinary skill in the art without departing from the scope and spirit of the embodiments illustrated. The selection of terms used herein is intended to best explain the principles, practical applications, or improvements to the techniques in the market of the embodiments, or to enable others of ordinary skill in the art to understand the embodiments disclosed herein.

1. An X-ray image processing method, comprising:
performing a plurality of exposures on at least one body part of a target subject by using an X-ray imaging system to obtain a plurality of first X-ray sub-images; and
performing image post-processing on the plurality of first X-ray sub-images to obtain a medical image, wherein the image post-processing comprises separately processing the plurality of first X-ray sub-images by using a virtual grid and performing image stitching, or comprises performing image stitching on the plurality of first X-ray sub-images and processing a stitched image by using a virtual grid, and the virtual grid is used to filter out noise caused by scattered radiation in the images.

2. The method according to claim 1, wherein the using a virtual grid includes processing the first X-ray sub-images or the stitched image based on a scatter image, wherein the scatter image is obtained by performing Fourier transform filtering on the first X-ray sub-images or the stitched image.

3. The method according to claim 2, wherein the using a virtual grid includes adjusting, based on an adjustment parameter, contrast of an image obtained through processing by using the virtual grid.

4. The method according to claim 1, wherein the image post-processing includes:
separately performing exposure amount adjustment and downsampling on each first X-ray sub-image to obtain a corresponding second X-ray sub-image, and then separately processing the plurality of second X-ray sub-images by using a virtual grid and performing image stitching, or performing image stitching on the plurality of second X-ray sub-images and processing a stitched image by using a virtual grid.

5. The method according to claim 4, wherein the image post-processing includes:
separately performing image enhancement on the stitched image and each second X-ray sub-image to obtain the medical image.

6. The method according to claim 5, wherein the image enhancement includes at least one of vertical equalization, multi-resolution enhancement, tissue equalization, inverse gamma distribution transformation, smart windowing, and lookup table mapping.

7. The method according to claim 1, wherein after the performing image stitching on the plurality of first X-ray sub-images and processing a stitched image by using a virtual grid, the image post-processing further comprises performing denoising on the plurality of first X-ray sub-images based on a scatter image, wherein the scatter image is obtained by performing Fourier transform filtering on the stitched image.

8. The method according to claim 7, wherein the denoising includes:
removing, based on a relationship between any second X-ray sub-image and a first region that is in the stitched image and that corresponds to the second X-ray sub-image, and a second region that is in the scatter image and that corresponds to the second X-ray sub-image, noise caused by scattered radiation in the second X-ray sub-image, wherein the second X-ray sub-image is obtained by performing exposure amount adjustment and downsampling on the first X-ray sub-images.

9. An X-ray image processing apparatus, comprising:
a first obtaining module, configured to perform a plurality of exposures on at least one body part of a target subject by using an X-ray imaging system to obtain a plurality of first X-ray sub-images; and
a second obtaining module, configured to perform image post-processing on the plurality of first X-ray sub-images to obtain a medical image, wherein the image post-processing comprises separately processing the plurality of first X-ray sub-images by using a virtual grid and performing image stitching, or comprises performing image stitching on the plurality of first X-ray sub-images and processing a stitched image by using a virtual grid, and the virtual grid is used to filter out noise caused by scattered radiation in the images.
